(19) United States
(12) Patent Application Publication
(10) Pub. No.: US 2020/0077915 A1
(43) Pub. Date: Mar. 12, 2020

(54) Massage Machine

(71) Applicant: Fuji Medical Instruments Mfg. Co., Ltd., Osaka (JP)

(72) Inventors: Masafumi TAKARABE, Osaka (JP); Masaaki MASUDA, Osaka (JP)

(73) Assignee: Fuji Medical Instruments Mfg. Co., Ltd., Osaka (JP)

(21) Appl. No.: 16/516,142

(22) Filed: Jul. 18, 2019

(30) Foreign Application Priority Data
Sep. 6, 2018 (JP) 2018-166944

(51) Int. Cl. A61B 5/0476; A61B 5/16; A61H 15/00

(52) U.S. Cl. CPC A61B 5/0476; A61B 5/4836; A61H 15/0078; A61B 5/165

(57) ABSTRACT

A massage machine includes an electroencephalograph (brain wave measuring device), arranged to measure brain waves of a person being treated, an emotion estimating portion, which, when a massage is being executed, estimates, based on the brain waves measured by the electroencephalograph, an emotion of the person being treated, a question message outputting portion, arranged to vocally output a question message for asking the person being treated whether or not to perform, on the massage currently being executed, a setting change that is in accordance with the emotion of the person being treated estimated by the emotion estimating portion, and a setting changing portion, performing the setting change if a reply of the person being treated to the question message is one that accepts the setting change.

### RELATED APPLICATIONS

This application claims priority to Japanese Patent Application No. 2018-166944 filed Sep. 6, 2018, which is hereby incorporated herein by reference in its entirety.

### BACKGROUND OF THE INVENTION

### 1. Field of the Invention

The present invention relates to a massage machine.

### 2. Description of the Related Art

Japanese Patent Application Publication No. 2003-275263 discloses analyzing biological information, constituted of blood pressure value, brain waves, body temperature, heart rate, body fat percentage, and body weight, of a person being treated and searching for a specific massage program from a plurality of massage programs based on the analysis results.

Japanese Patent Application Publication No. 2015-229040 discloses comparing brain waves, measured by an electroencephalograph, with characteristic patterns, acquired in advance for each of a plurality of emotions (for example, “like,” “interested,” “stress,” “sleepiness,” and “concentrate”), to estimate strengths (degrees) of the respective emotions of a subject.

### SUMMARY OF THE INVENTION

An object of the present invention is to provide a massage machine, with which a setting change that is in accordance with an emotion of a person being treated can be performed readily on a massage currently being executed.

In order to overcome the previously unrecognized and unsolved challenges described above, a preferred embodiment of the present invention provides a massage machine. The massage machine includes a brain wave measuring device, arranged to measure brain waves of a person being treated, an emotion estimating portion, which, when a massage is being executed, estimates, based on the brain waves measured by the brain wave measuring device, an emotion of the person being treated, a question message outputting portion, arranged to vocally output a question message for asking the person being treated whether or not to perform, on the massage currently being executed, a setting change that is in accordance with the emotion of the person being treated estimated by the emotion estimating portion, and a setting changing portion, performing the setting change if a reply of the person being treated to the question message is one that accepts the setting change.

According to this arrangement, a massage machine, with which a setting change that is in accordance with an emotion of a person being treated can be performed readily on a massage currently being executed, can be realized.

In the preferred embodiment of the present invention, the setting change that is in accordance with the emotion of the person being treated includes a setting change of a strength of the massage currently being executed.

In the preferred embodiment of the present invention, the setting change that is in accordance with the emotion of the person being treated includes a setting change of a massaging time of the massage currently being executed.

In the preferred embodiment of the present invention, the setting change that is in accordance with the emotion of the person being treated includes a setting change related to whether or not to play music with the massage currently being executed.

In the preferred embodiment of the present invention, the question message includes a content suggesting the emotion estimated by the emotion estimating portion.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a partially cutaway perspective view of an outer appearance of a chair type massage machine to which a massage machine according to a preferred embodiment of the present invention is applied.

FIG. 2 is an illustrative perspective view of an arrangement of a massage unit.

FIG. 3 is a block diagram of an electrical configuration of a headset device.

FIG. 4 is a block diagram of an electrical configuration of a tablet PC.

FIG. 5 is a block diagram of an electrical configuration of the massage machine.

FIG. 6 is a flowchart of operations of a setting change controller of the tablet PC.

FIG. 7 is a flowchart of operations of a setting change process portion inside a main controller.

FIG. 8 is a table of specific examples of emotions of a person being treated that are estimated by an emotion estimating portion, contents of corresponding question messages, replies of the person being treated to the question messages, and setting change operations and response messages corresponding to the replies of the person being treated.

### DETAILED DESCRIPTION OF THE PREFERRED EMBODIMENTS

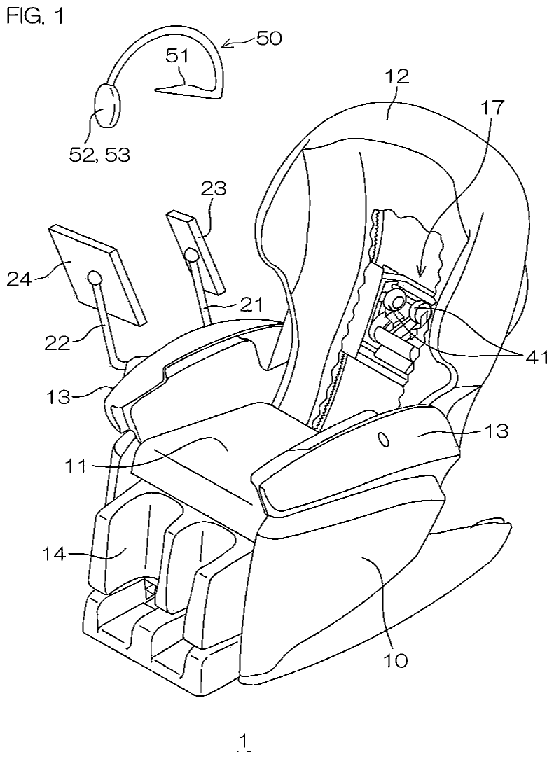

FIG. 1 is a partially cutaway perspective view of an outer appearance of a chair type massage machine to which a massage machine according to a preferred embodiment of the present invention is applied.

The chair type massage machine 1 (hereinafter referred to as the “massage machine 1”) includes a seat portion 11, a backrest portion 12, armrest portions 13, a footrest portion (ottoman) 14, and a base portion 10, serving as a foundation for these portions.

In the description that follows, a front/rear direction, a right/left direction, and an up/down direction shall respectively refer to a front/rear direction, a right/left direction, and an up/down direction as viewed by a person being treated when the person being treated is seated in an ordinary orientation on the massage machine 1.

The seat portion 11 is disposed on the base portion 10. The backrest portion 12 is disposed at a rear of the seat portion 11. The armrest portions 13 are disposed at both right and left sides of the seat portion 11. The footrest portion 14 is disposed at a front side of the seat portion 11. The backrest portion 12 is supported by a backrest pivoting actuator 15 (see FIG. 5) such as to be tiltable with respect to the seat portion 11. Also, the footrest portion 14 is enabled to be pivoted around a support shaft, provided at a vicinity of a seat portion upper portion and extending in the right/left direction, as a center by a footrest portion pivoting actuator 16 (see FIG. 5).

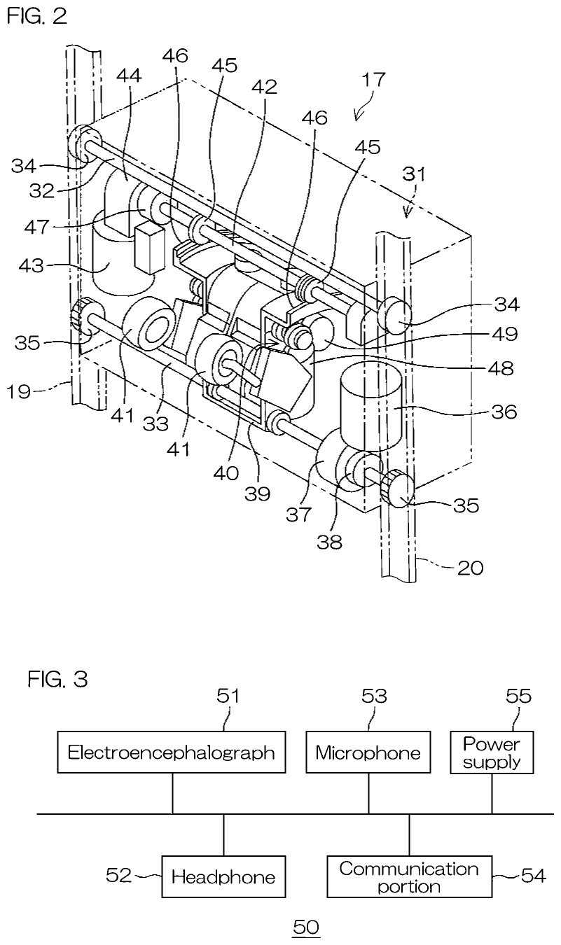

A massage unit 17 is incorporated in the backrest portion 12. The massage unit 17 is arranged to perform various types of massage using a pair of right and left massage elements (kneading balls) 41. The backrest portion 12 is provided with a pair of right and left guide rails 19 and 20 (see FIG. 2) of U-shaped cross section extending in the up/down direction, and the massage unit 17 is enabled to move in the up/down direction along the guide rails 19 and 20. A detailed arrangement of the massage unit 17 shall be described later.

The seat portion 11, the backrest portion 12, the armrest portions 13, and the footrest portion 14 are provided with airbags (not shown). Each airbag is inflated by being supplied with air from an air pump (not shown) via a solenoid valve (not shown). Each air bag has a flat form when deflated and is suitably inflated to apply a pressing stimulus to the person to be treated.

An operating device supporting member 21 and a tablet supporting member 22 are mounted to one of the armrest portions 13. An operating device (remote controller) 23, for operation of the massage machine 1 by the person being treated, is detachably mounted to the operating device supporting member 21. A tablet type computer (hereinafter referred to as the “tablet PC”) 24 is detachably mounted to the tablet supporting member 22.

The massage machine 1 further includes a headset device 50 mounted on a head portion of the person being treated. The headset device 50 includes an electroencephalograph (brain wave measuring device) 51, a headphone 52, and a microphone 53.

FIG. 2 is an illustrative perspective view of the arrangement of the massage unit 17.

The massage unit 17 is mounted vertically movably mounted to the guide rails 19 and 20. The massage unit 17 includes a main frame 31 of rectangular frame shape. The main frame 31 is constituted of a pair of right and left side walls and a top wall and a bottom wall respectively joining mutual upper ends and mutual lower ends of the side walls. A guide shaft 32 and a raising/lowering driveshaft 33 that extend in the right/left direction are rotatably mounted to the main frame 31.

The guide shaft 32 is disposed at an upper portion of the main frame 31 and the raising/lowering driveshaft 33 is mounted to a lower portion of the main frame 31. Both end portions of the guide shaft 32 and the raising/lowering driveshaft 33 project outward from both side walls of the main frame 31. Guide rollers 34, guided by the guide rails 19 and 20, are mounted to both end portions of the guide shaft 32. Pinion gears 35, engaging with racks (not shown) provided in the guide rails 19 and 20, are mounted to both end portions of the raising/lowering driveshaft 33.

A raising/lowering motor 36, arranged to rotate the raising/lowering driveshaft 33, is mounted to the main frame 31. The raising/lowering motor 36 is coupled to the raising/lowering driveshaft 33 via a gear mechanism 37. The massage unit 17 is raised and lowered along the guide rails 19 and 20 by the raising/lowering motor 36 being rotated.

The massage unit 17 is provided with a raising/lowering position sensor 38 arranged to detect a rotation amount of the raising/lowering driveshaft 33 to detect a raising/lowering position (up/down direction position) of the massage unit 17. The raising/lowering position sensor 38 is constituted of a rotary encoder arranged to detect the rotation amount of the raising/lowering driveshaft 33.

At an intermediate portion of a length of the raising/lowering driveshaft 33, a swinging frame 39 is mounted such as to be swingable in the front/rear direction. A pair of right and left massage elements 41 and a massage element driving unit 40, including a driving mechanism therefor, are mounted to the swinging frame 39. The main frame 31 is provided with a massage element driving unit advancing/retreating mechanism (moving mechanism) arranged to make the swinging frame (massage element driving unit) advance and retreat in the front/rear direction.

The massage element driving unit advancing/retreating mechanism shall now be described. An advancing/retreating shaft 42, disposed at a lower side of the guide shaft 32 and extending in the right/left direction, is rotatably mounted to the main frame 31. An advancing/retreating motor 43, arranged to rotate the advancing/retreating shaft 42, is mounted to a side portion at one side of the main frame 31. The advancing/retreating motor 43 is coupled to the advancing/retreating shaft 42 via a gear mechanism 44.

A pair of right and left pinion gears 45 are mounted to intermediate portions of a length of the advancing/retreating shaft 42. Arcuate racks 46, engaging with the pair of right and left pinion gears 45, are respectively provided at upper portions of the swinging frame 39. When the advancing/retreating shaft 42 is rotated by the advancing/retreating motor 43, the pinion gears 45 rotate and the arcuate racks 46 move. Thereby, the swinging frame 39 swings around the raising/lowering driveshaft 33 as a center. Thereby, the massage element driving unit 40 (massage elements 41) advances and retreats in the front/rear direction.

The massage unit 17 is provided with a front/rear position sensor 47 arranged to detect a rotation amount of the advancing/retreating shaft 42 to detect a front/rear position of the massage element driving unit 40. The front/rear position sensor 47 is constituted of an encoder arranged to detect the rotation amount of the advancing/retreating shaft 42.

The massage element driving unit 40 includes a kneading mechanism that eccentrically pivots the massage elements 41 to perform a kneading operation and a tapping mechanism that swings the massage elements 41 back and forth to perform a tapping operation. The kneading mechanism includes a kneading motor 48 as an actuator (driving source). The tapping mechanism includes a tapping motor 49 as an actuator (driving source).

FIG. 3 is a block diagram of an electrical configuration of the headset device 50.

The headset device 50 includes the electroencephalograph 51, the headphone 52, the microphone 53, a communication portion 54, and a power supply 55 arranged to supply electric power to these components.

The electroencephalograph 51 is a device arranged to measure brain waves of the person being treated.

The headphone 52 is a voice outputting device arranged to vocally output a voice message, etc., transmitted from the tablet PC 24.

The microphone 53 is a sound collecting device for collecting the voice of the person being treated.

The communication portion 54 is a communication interface arranged to transmit the brain waves measured by the electroencephalograph 51 and the voice collected by the microphone 53 to the tablet PC 24 and to receive voice signals from the tablet PC 24 and transmit the signals to the headphone 52. Although in the present preferred embodiment, the transmission and reception of data to and from the headset device 50 are performed wirelessly, these may be performed by wire instead.

When the power of the headset device 50 is turned on, the electroencephalograph 51 measures the brain waves of the person being treated on whom the headset device 50 is mounted. The brain waves measured by the electroencephalograph 51 are transmitted to the tablet PC 24 by the communication portion 54.

When the power of the headset device 50 is turned on, the microphone 53 collects the voice of the person being treated. The voice collected by the microphone 53 is transmitted to the tablet PC 24 by the communication portion 54.

When a voice message from the tablet PC 24 is received by the communication portion 54 when the power of the headset device 50 is turned on, the received voice message is vocally output from the headphone 52.

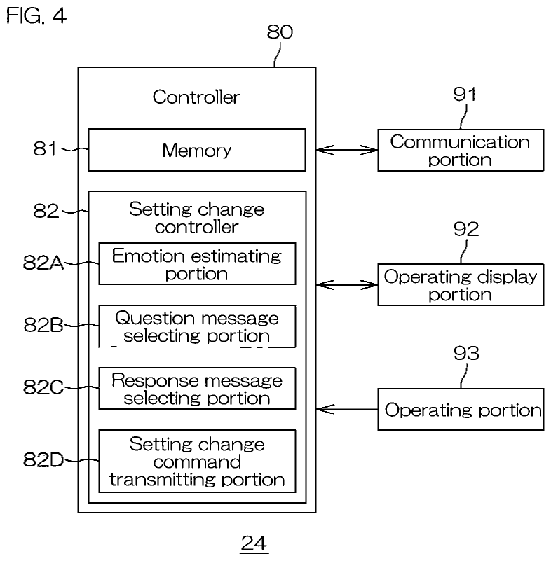

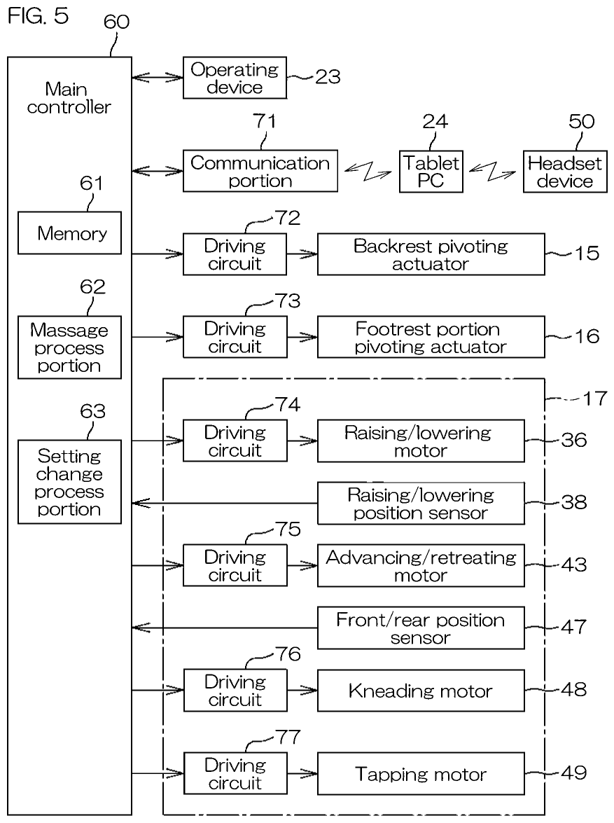

FIG. 4 is a block diagram of an electrical configuration of the tablet PC 24.

The tablet PC 24 includes a controller 80. The controller 80 includes a microcomputer and includes a CPU and a memory (RAM, ROM, or nonvolatile memory) 81, etc. A setting change control program for performing setting change control of massage based on the brain waves measured by the electroencephalograph 51, a plurality of types of question messages and a plurality of types of response messages used when executing the setting change control program, etc., are stored in the memory 81.

A communication portion 91, an operating display portion 92, an operating portion 93, etc., are connected to the controller 80. The communication portion 91 is a communication interface arranged to perform wireless communication with the headset device 50 and perform wireless communication with a main controller 60 (see FIG. 5) of the massage machine 1 to be described below.

The operating display portion 92 is constituted, for example, of a touch panel type liquid crystal display. The operating portion 93 is constituted of one or a plurality of switches.

The controller 80 includes a setting change controller 82 arranged to perform a setting change control process in accordance with the setting change control program. The setting change controller 82 includes an emotion estimating portion 82A, a question message selecting portion 82B, a response message selecting portion 82C, and a setting change command transmitting portion 82D.

Based on the brain waves measured by the electroencephalograph 51 when the massage is being executed, the emotion estimating portion 82A estimates an emotion of the person being treated.

The question message selecting portion 82B selects, from among the plurality of types of question messages stored in the memory 81, a question message for asking the person being treated whether or not to perform, on the massage currently being executed, a setting change that is in accordance with the emotion of the person being treated estimated by the emotion estimating portion 82A and transmits the selected message to the headset device 50. The question message is constituted of a voice message. The question message transmitted to the headset device 50 is vocally output by the headphone 52.

The question message preferably includes a content that suggests the emotion estimated by the emotion estimating portion 82A. The question message selecting portion 82B and the headphone 52 constitute an example of a question message outputting portion of the present invention.

When a reply of the person being treated to a question message is received, the response message selecting portion 82C selects, from among the plurality of types of response messages stored in the memory 81, a response message to the reply of the person being treated and transmits the selected message to the headset device 50. The response message is constituted of a voice message. The response message transmitted to the headset device 50 is vocally output by the headphone 52.

If the reply of the person being treated to the question message is one that accepts a setting change, the setting change command transmitting portion 82D transmits a command (setting change command) for executing the setting change to the main controller 60 of the massage machine 1. The setting change command transmitting portion 82D and a below-described setting change process portion 63 (see FIG. 5) inside the main controller 60 constitute an example of a setting changing portion of the present invention.

FIG. 5 is a block diagram of an electrical configuration of the massage machine 1. In FIG. 5, the air pump, a driving circuit therefor, the solenoid valve, and a driving circuit therefor that are arranged to inflate and deflate each air bag are omitted for convenience of description.

The main controller 60, arranged to control the massage machine 1, is incorporated in an interior of the massage machine 1. The main controller 60 includes a microcomputer and includes a CPU and a memory (RAM, ROM, or nonvolatile memory) 61, etc. A program for controlling the massage machine 1 and necessary data, etc., are stored in the memory 61. Also, the main controller 60 includes an internal clock.

The operating device 23, a communication portion 71, a driving circuit 72 of the backrest pivoting actuator 15, and a driving circuit 73 of the footrest portion pivoting actuator 16 are connected to the main controller 60. Further, a driving circuit 74 of the raising/lowering motor 36, a driving circuit 75 of the advancing/retreating motor 43, a driving circuit 76 of the kneading motor 48, and a driving circuit 77 of the tapping motor 49 inside the massage unit 17 are connected to the main controller 60. Further, the raising/lowering position sensor 38 and the front/rear position sensor 47 inside the massage unit 17 are connected to the main controller 60.

The communication portion 71 is a communication interface arranged to perform wireless communication with the tablet PC 24. As described above, the tablet PC 24 is wirelessly communicable with the headset device 50.

The main controller 60 controls the driving circuits 72 and 73 of the respective actuators 15 and 16 based on operations of the operating device 23, etc. Also, the main controller 60 controls the driving circuits 74 to 77 of the respective motors 36, 43, 48, and 49, the driving circuit (not shown) of the air pump, and the driving circuit (not shown) of the solenoid valve based on operations of the operating device 23, a setting change command from the tablet PC 24, etc. The massage machine 1 is thereby made capable of performing various types of massage.

With a massage mode of the massage machine 1, there is an automatic mode and a manual mode. In the automatic mode, massage is performed in accordance with a massage course selected from among a plurality of types of massage courses by the person being treated. In the manual mode, the massage is performed in accordance with a massage type selected by the person being treated. A plurality of types of massage programs that are in accordance with the plurality of types of massage courses are stored in the memory 61.

The main controller 60 includes a massage process portion 62 and the setting change process portion 63. The massage process portion 62 is a process portion, which, as is well known, performs the massage process in accordance with the massage mode (automatic mode or manual mode) and the massage course selected by the person being treated. The setting change process portion 63 is a process portion that performs a setting change process based on the setting change command from tablet PC 24.

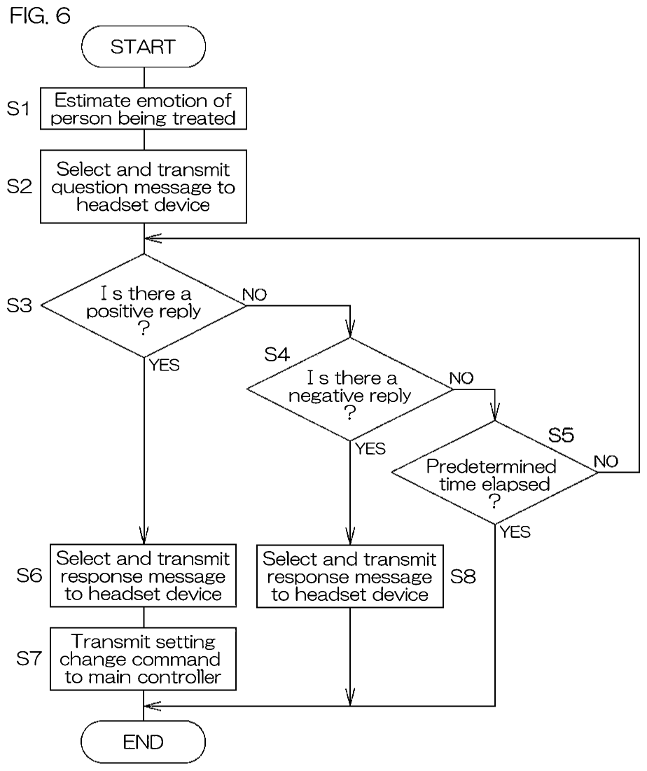

FIG. 6 is a flowchart of operations of the setting change controller 82 of the tablet PC 24. The process of FIG. 6 is executed repeatedly in each predetermined calculation cycle when a massage is being performed by the massage machine 1.

The emotion estimating portion 82A inside the setting change controller 82 estimates the emotion of the person being treated based on the brain waves of the person being treated measured by the electroencephalograph 51 (step S1). The specific content of the emotion of the person being treated estimated in step S1 shall be described below.

Next, the question message selecting portion 82B inside the setting change controller 82 selects, from among the plurality of types of question messages stored in the memory 81, the question message for asking the person being treated whether or not to perform the setting change that is in accordance with the emotion of the person being treated estimated in step S1 and transmits the selected message to the headset device 50 (step S2). The question message selected by the question message selecting portion 82B is thereby vocally output from the headphone 52.

The person being treated makes a positive reply of “yes” or a negative reply of “no” to the question message vocally output from the headphone 52.

The setting change controller 82 monitors whether or not the person being treated made the positive reply (step S3), whether or not the person being treated made the negative reply (step S4), and whether or not a predetermined time has elapsed since transmitting the question message in step S2 (step S5).

If it is determined that the positive reply is made to the question message (step S3: YES), the setting change controller 82 performs changeover to step S6. In step S6, the response message selecting portion 82C inside the setting change controller 82 selects, from among the plurality of types of response messages stored in the memory 81, a response message corresponding to the positive reply to the present question message and transmits the selected message to the headset device 50 (step S6). The response message selected by the response message selecting portion 82C is thereby vocally output from the headphone 52.

Also, if it is determined that the positive reply is made to the question message, the setting change command transmitting portion 82D inside the setting change controller 82 transmits a command (setting change command) for executing the setting change corresponding to the present question message to the main controller 60 (step S7). Thereby, the main controller 60 is made to execute the setting change corresponding to the present question message. The setting change controller 82 then ends the process of the present calculation cycle.

If it is determined that the negative reply is made to the question message (step S4: YES), the setting change controller 82 performs changeover to step S8. In step S8, the response message selecting portion 82C inside the setting change controller 82 selects, from among the plurality of types of response messages stored in the memory 81, a response message corresponding to the negative reply to the present question message and transmits the selected message to the headset device 50. The response message selected by the response message selecting portion 82C is thereby vocally output from the headphone 52. The setting change controller 82 then ends the process of the present calculation cycle.

If the predetermined time elapses without the positive reply or the negative reply being made to the question message (step S5: YES), the setting change controller 82 ends the process of the present calculation cycle.

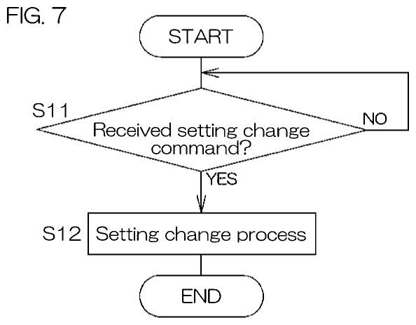

FIG. 7 is a flowchart of operations of the setting change process portion 63 inside the main controller 60.

Upon receiving a setting change command from the tablet PC 24 (step S11: YES), the setting change process portion 63 performs the setting change process that is in accordance with the received setting change command (step S12).

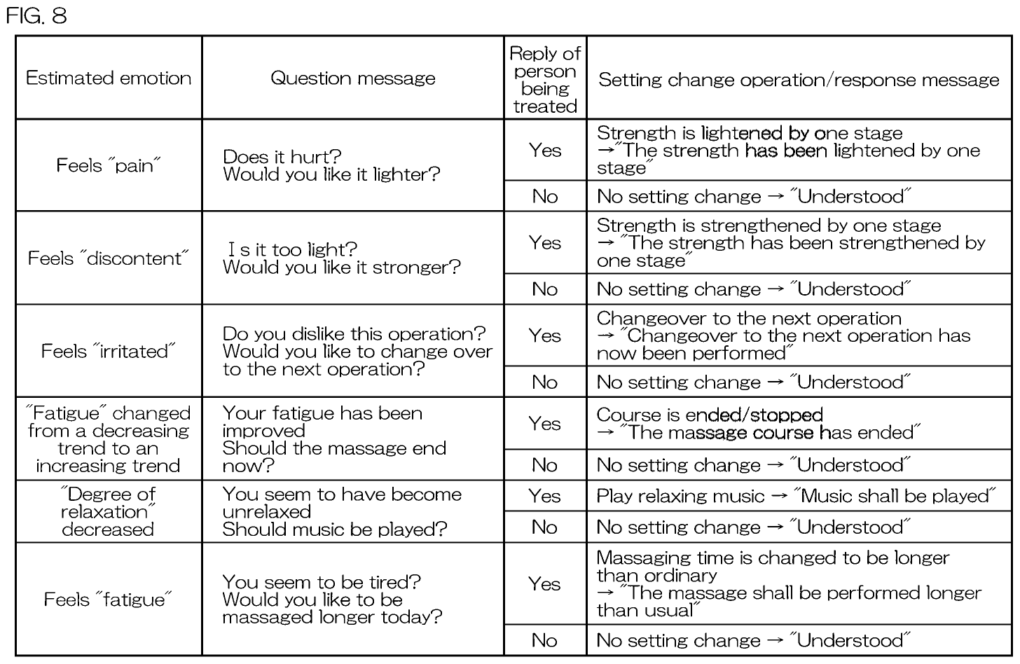

FIG. 8 is a table of specific examples of emotions of the person being treated that are estimated by the emotion estimating portion, contents of the corresponding question messages, replies (positive reply or negative reply) of the person being treated to the question messages, and setting change operations and response messages corresponding to the replies of the person being treated.

In the example of FIG. 8, it is estimated in step S1 whether or not the person being treated is feeling “pain,” whether or not the person being treated is feeling “discontent,” whether or not the person being treated is feeling “irritated,” whether or not a “fatigue” that the person being treated is feeling changed from a decreasing trend to an increasing trend, whether or not a “degree of relaxation” that the person being treated is feeling decreased, whether or not the person being treated is feeling “fatigue,” etc.

If it is estimated that the person being treated is feeling “pain,” a question message, for example, of: “Does it hurt? Would you like it lighter?” is vocally output (see step S2). If the person being treated replies “Yes” to this question message, a strength of the massage currently being performed is lightened by one stage and a response message, for example, of: “The strength has been lightened by one stage.” is vocally output (see steps S3, S6, and S7).

On the other hand, if the person being treated replies “No” to the question message, the setting change operations are not performed and a response message, for example, of: “Understood.” is vocally output (see steps S4 and S8). The operation for the case where the person being treated replies “No” to a question message is also the same for other question messages.

If it is estimated that the person being treated is feeling “discontent,” a question message, for example, of: “Is it too light? Would you like it stronger?” is vocally output (see step S2). If the person being treated replies “Yes” to this question message, the strength of the massage currently being performed is strengthened by one stage and a response message, for example, of: “The strength has been strengthened by one stage.” is vocally output (see steps S3, S6, and S7).

If it is estimated that the person being treated is feeling “irritated,” a question message, for example, of: “Do you dislike this operation? Would you like to change over to the next operation?” is vocally output (see step S2). If the person being treated replies “Yes” to this question message, a changeover to the next operation (operation of the next massage type) is performed and a response message, for example, of: “Changeover to the next operation has now been performed.” is vocally output (see steps S3, S6, and S7).

If it is estimated that the “fatigue” that the person being treated is feeling changed from a decreasing trend to an increasing trend, a question message, for example, of: “Your fatigue has been improved. Should the massage end now?” is vocally output (see step S2). If the person being treated replies “Yes” to this question message, the massage course currently being performed is stopped and a response message, for example, of: “The massage course has ended.” is vocally output (see steps S3, S6, and S7).

If it is estimated that the “degree of relaxation” that the person being treated is feeling decreased, a question message, for example, of: “You seem to have become unrelaxed. Should music be played?” is vocally output (see step S2). If the person being treated replies “Yes” to this question message, relaxing music is played and a response message, for example, of: “Music shall be played.” is vocally output (see steps S3, S6, and S7). In this case, although audio data of relaxing music may be transmitted from the main controller 60 side to the headset device 50 via the tablet PC 24, audio data of relaxing music stored in advance at the tablet PC 24 side may be transmitted to the headset device 50 instead.

If it is estimated that the person being treated is feeling “fatigue,” a question message, for example, of: “You seem to be tired? Would you like to be massaged longer today?” is vocally output (see step S2). If the person being treated replies “Yes” to this question message, a massaging time is changed to be longer than ordinary and a response message, for example, of: “The massage shall be performed longer than usual.” is vocally output (see steps S3, S6, and S7).

The types of emotions estimated by the emotion estimating portion 82A are not limited to the types of emotions mentioned above. Similarly, the question messages for the emotions estimated by the emotion estimating portion 82A are not limited to the question messages mentioned above. Similarly, the response messages to the replies of the person being treated are not limited to the response messages mentioned above.

By the preferred embodiment described above, a massage machine, with which a setting change that is in accordance with (that is suited to) an emotion of a person being treated can be performed readily on a massage currently being executed, can be realized.

Although a preferred embodiment of the present invention has been described above, the present invention may also be implemented in yet other modes. For example, although in the preferred embodiment described above, the headphone 52 and the microphone 53 are provided in the headset device 50 that includes the electroencephalograph 51, the headphone 52 and the microphone 53 may be provided in the tablet PC 24 instead. In this case, the headphone 52 and the microphone 53 are not provided in the headset device 50.

Also, although in the preferred embodiment described above, the massage machine 1 includes the tablet PC 24 in addition to the main controller 60, the functions of the tablet PC 24 may instead be included in the main controller 60.

Also, although in the preferred embodiment described above, a case where the present invention is applied to a chair type massage machine was described, the present invention may be applied to a massage machine other than a chair type (for example, a seat type massage machine) as long as it is a massage machine that includes a tapping mechanism or a kneading mechanism having an electric motor as a driving source. Also, the present invention may be applied to a massage machine that does not include an air bag.

The present application corresponds to Japanese Patent Application No. 2018-166944 filed on Sep. 6, 2018 in the Japan Patent Office, and the entire disclosure of this application is incorporated herein by reference.

While preferred embodiments of the present invention have been described above, it is to be understood that variations and modifications will be apparent to those skilled in the art without departing the scope and spirit of the present invention. The scope of the present invention, therefore, is to be determined solely by the following claims.

What is claimed is:

1. A massage machine comprising:
a brain wave measuring device, arranged to measure brain waves of a person being treated;
an emotion estimating portion, which, when a massage is being executed, estimates, based on the brain waves measured by the brain wave measuring device, an emotion of the person being treated;
a question message outputting portion, arranged to vocally output a question message for asking the person being treated whether or not to perform, on the massage currently being executed, a setting change that is in accordance with the emotion of the person being treated estimated by the emotion estimating portion; and
a setting changing portion, performing the setting change if a reply of the person being treated to the question message is one that accepts the setting change.

2. The massage machine according to claim 1, wherein the setting change that is in accordance with the emotion of the person being treated includes a setting change of a strength of the massage currently being executed.

3. The massage machine according to claim 1, wherein the setting change that is in accordance with the emotion of the person being treated includes a setting change of a massaging time of the massage currently being executed.

4. The massage machine according to claim 1, wherein the setting change that is in accordance with the emotion of the person being treated includes a setting change related to whether or not to play music with the massage currently being executed.

5. The massage machine according to claim 1, wherein the question message includes a content suggesting the emotion estimated by the emotion estimating portion.

6. The massage machine according to claim 2, wherein the setting change that is in accordance with the emotion of the person being treated includes a setting change of a massaging time of the massage currently being executed.

7. The massage machine according to claim 2, wherein the setting change that is in accordance with the emotion of the person being treated includes a setting change related to whether or not to play music with the massage currently being executed.

8. The massage machine according to claim 3, wherein the setting change that is in accordance with the emotion of the person being treated includes a setting change related to whether or not to play music with the massage currently being executed.

9. The massage machine according to claim 6, wherein the setting change that is in accordance with the emotion of the person being treated includes a setting change related to whether or not to play music with the massage currently being executed.
